(19) United States
(12) Patent Application Publication
(10) Pub. No.: US 2019/0189291 A1
(43) Pub. Date: Jun. 20, 2019

(54) ACTIVITY NOTIFICATION AGGREGATION IN A ROLE-SPECIFIC FEED

(71) Applicant: salesforce.com, inc., San Francisco, CA (US)

(72) Inventors: ROBERT BATTERTON, STILLWATER, MN (US); SUSAN LUCAS COLLINS, ALPHARETTA, GA (US)

(73) Assignee: salesforce.com, inc., San Francisco, CA (US)

(21) Appl. No.: 15/845,755

(22) Filed: Dec. 18, 2017

(51) Int. Cl. G16H 80/00; G06F 17/30; G06Q 50/00

(52) U.S. Cl. CPC G16H 80/00; G06Q 50/01; G06F 17/30994; G06F 17/30498

(57) ABSTRACT

A method of providing role specific notification views in an enterprise social networking system comprises receiving a connection request for a persona; retrieving one or more case team assignments for the persona; for each case team assignment for the persona, adding the persona to the case team for the assignment; for each case team assignment for the persona, retrieving a role-specific view for the persona of a viewing log associated with the assigned case team wherein the viewing log provides a view of events that pertains to members of the case team and that occur while the members are members of the case team wherein the role-specific view for the persona provides a view of events from the viewing log that occur while the persona is a member of the case team; and providing a notification view that includes the role-specific view for each case team assignment for the persona.

### TECHNICAL FIELD

Embodiments of the subject matter described herein relate generally to computer systems and enterprise social networking systems, and more particularly to techniques for customizing information feeds from enterprise social networking systems.

### BACKGROUND

An enterprise social networking system can provide users with an information feed of events tracked by the enterprise social networking system. Social networking system users can track project and sales opportunities, solicit input from knowledgeable colleagues, and otherwise enrich the social networking experience through updates about coworkers, customers, topics of interest, and business objects. Users can also form groups to facilitate collaboration between group members in the enterprise.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a block diagram of an example social networking system that may be implemented in the context of an example multi-tenant system, in accordance with various embodiments.

FIG. 2 is a block diagram of an example computing environment that can be used to implement an enterprise social networking system, in accordance with various embodiments.

FIG. 3A is a diagram depicting example personas, devices, and notification targets in an example patient care enterprise environment in which an example enterprise social networking system may be used, in accordance with various embodiments.

FIG. 3B is a diagram illustrating user group specific virtual logs that can be employed with example personas, devices, and notification targets in an example patient care enterprise environment to track associated events, in accordance with various embodiments.

FIG. 4 is a relationship diagram illustrating an example relationship between events, an enterprise log, and user group specific virtual logs, in accordance with various embodiments.

FIG. 5 is a block diagram depicting an example enterprise networking system for generating group virtual logs and case team virtual logs from which a notification feed can be provided, in accordance with various embodiments.

FIG. 6 is a process flow chart depicting an example process for adding or removing members from a case team in an enterprise networking system, in accordance with various embodiments.

FIG. 7 is a process flow chart depicting an example process in an enterprise networking system for providing a targeted feed for a persona, in accordance with various embodiments.

FIG. 8 is a process flow chart depicting another example process in an enterprise networking system for providing a targeted feed for a persona, in accordance with various embodiments.

### DETAILED DESCRIPTION

Disclosed herein are systems and methods for customizing an information feed in an enterprise networking system. The subject matter described herein discloses apparatus, systems, techniques and articles for focusing an information feed on feed items that may be more relevant to a subscriber of the information feed.

The following disclosure provides many different embodiments, or examples, for implementing different features of the provided subject matter. The following detailed description is merely exemplary in nature and is not intended to limit the invention or the application and uses of the invention. Furthermore, there is no intention to be bound by any theory presented in the preceding background or the following detailed description.

Social networking systems can be used to facilitate communication among social networking system users who may be individual people and/or groups of people. Social network systems may be implemented for various organizations such as business entities, business partnerships, academic institutions, groups or departments within an organization, and others. Social networking systems can provide users with access to one or more information feeds that include information updates presented as feed items or entries in the feed. A feed item may include a single information update or a collection of individual information updates. A feed item can include various types of data including character-based data, audio data, image data and/or video data. An information feed can be displayed in a graphical user interface (GUI) on a display device such as the display of a computing device. The information updates can include various social network data from various sources and can be stored in an on-demand database service environment. Feed items can be stored and maintained in one or more database tables, e.g., as rows in the table(s), that can be accessed to retrieve relevant information to be presented as part of a displayed information feed. A social networking system may be implemented in a multi-tenant database environment, although such an environment is not required in all embodiments.

A social networking system may allow a user to follow data objects in the form of records such as cases, accounts, or opportunities, in addition to following individual users and groups of users. The “following” of a record stored in a database allows a user to track the progress of that record. Updates to the record, also referred to herein as changes to the record, are one type of information update that can occur and be noted on an information feed such as a record feed or a news feed of a user subscribed to the record. Examples of record updates include field changes in the record, updates to the status of a record, as well as the creation of the record itself. Some records are publicly accessible, such that any user can follow the record, while other records are private, for which appropriate security clearance/permissions are a prerequisite to a user following the record.

Information updates can include various types of updates, which may or may not be linked with a particular record. For example, information updates can be user-submitted messages or can otherwise be generated in response to user actions or in response to events. Examples of messages include posts and comments.

Users can follow a record by subscribing to the record, as mentioned above. Users can also follow other entities such as other types of data objects, other users, and groups of users. Feed tracked updates regarding such entities are one type of information update that can be received and included in the user's information feed. Any number of users can follow a particular entity and thus view information updates pertaining to that entity on the users' respective information feeds.

An information feed may be specific to a group of users of an online social network. For instance, a group of users may publish an information feed. Members of the group may view and post to the group feed in accordance with a permissions configuration for the information feed and the group. Information updates in a group context can also include changes to group status information.

With the increased use of online social networking systems, a user's information feed may include a large number of feed items, potentially making it time consuming for a user to keep up with all feed items. Many of the feed items may be relevant to the user but many of the feed items may not be relevant. As a result, a user accessing the information feed may have to spend a great deal of time, energy, and effort to read through numerous feed items, identify feed items of interest, consume the content of selected information in the feed, and synthesize the information to mentally piece together a larger conversation defined by the relevant feed items. Disclosed herein are systems and methods for customizing the information feed to focus the information feed to feed items that may be more relevant to the user.

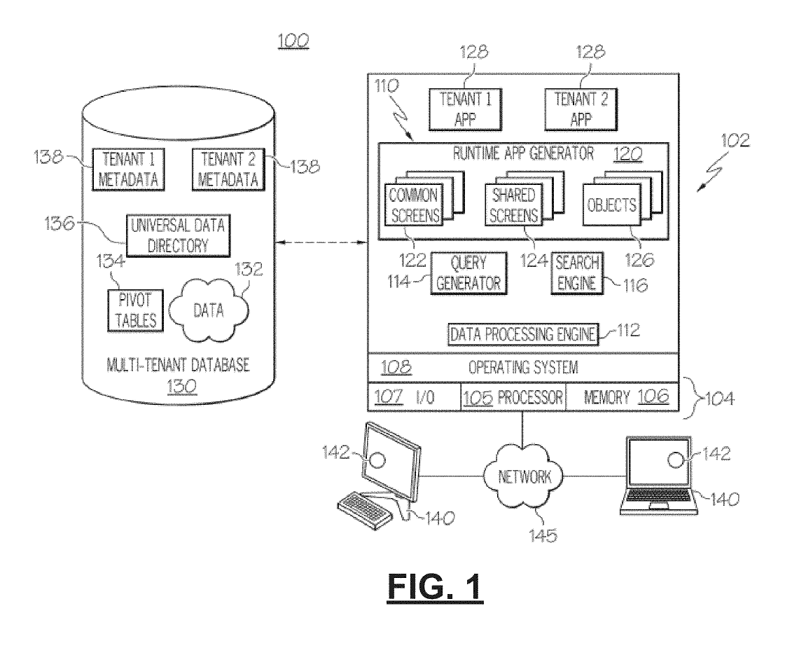

Turning now to FIG. 1, an exemplary social networking system may be implemented in the context of an example multi-tenant system 100. The example multi-tenant system 100 of FIG. 1 includes a server 102 that dynamically creates and supports virtual applications 128 based upon data 132 from a common database 130 that is shared between multiple tenants, alternatively referred to herein as a multi-tenant database. Data and services generated by the virtual applications 128 are provided via a network 145 to any number of client devices 140, as desired. Each virtual application 128 is suitably generated at run-time (or on-demand) using a common application platform 110 that securely provides access to the data 132 in the database 130 for each of the various tenants subscribing to the multi-tenant system 100.

As used herein, a “tenant” or an “organization” should be understood as referring to a group of one or more users or entities that shares access to common subset of the data within the multi-tenant database 130. In this regard, each tenant includes one or more users associated with, assigned to, or otherwise belonging to that respective tenant. To put it another way, each respective user within the multi-tenant system 100 is associated with, assigned to, or otherwise belongs to a particular tenant of the plurality of tenants supported by the multi-tenant system 100. Tenants may represent customers, customer departments, business or legal organizations, and/or any other entities that maintain data for particular sets of users within the multi-tenant system 100 (i.e., in the multi-tenant database 130). For example, the application server 102 may be associated with one or more tenants supported by the multi-tenant system 100. Although multiple tenants may share access to the server 102 and the database 130, the particular data and services provided from the server 102 to each tenant can be securely isolated from those provided to other tenants (e.g., by restricting other tenants from accessing a particular tenant's data using that tenant's unique organization identifier as a filtering criterion). The multi-tenant architecture therefore allows different sets of users to share functionality and hardware resources without necessarily sharing any of the data 132 belonging to or otherwise associated with other tenants.

The multi-tenant database 130 is any sort of repository or other data storage system capable of storing and managing the data 132 associated with any number of tenants. The database 130 may be implemented using any type of conventional database server hardware. In various embodiments, the database 130 shares processing hardware 104 with the server 102. In other embodiments, the database 130 is implemented using separate physical and/or virtual database server hardware that communicates with the server 102 to perform the various functions described herein. In an exemplary embodiment, the database 130 includes a database management system or other equivalent software capable of determining an optimal query plan for retrieving and providing a particular subset of the data 132 to an instance of virtual application 128 in response to a query initiated or otherwise provided by a virtual application 128. The multi-tenant database 130 may alternatively be referred to herein as an on-demand database, in that the multi-tenant database 130 provides (or is available to provide) data at run-time to on-demand virtual applications 128 generated by the application platform 110.

In practice, the data 132 may be organized and formatted in any manner to support the application platform 110. In various embodiments, the data 132 is suitably organized into a relatively small number of large data tables to maintain a semi-amorphous “heap”-type format. The data 132 can then be organized as needed for a particular virtual application 128. In various embodiments, conventional data relationships are established using any number of pivot tables 134 that establish indexing, uniqueness, relationships between entities, and/or other aspects of conventional database organization as desired. Further data manipulation and report formatting is generally performed at run-time using a variety of metadata constructs. Metadata within a universal data directory (UDD) 136, for example, can be used to describe any number of forms, reports, workflows, user access privileges, business logic and other constructs that are common to multiple tenants. Tenant-specific formatting, functions and other constructs may be maintained as tenant-specific metadata 138 for each tenant, as desired. Rather than forcing the data 132 into an inflexible global structure that is common to all tenants and applications, the database 130 is organized to be relatively amorphous, with the pivot tables 134 and the metadata 138 providing additional structure on an as-needed basis. To that end, the application platform 110 suitably uses the pivot tables 134 and/or the metadata 138 to generate “virtual” components of the virtual applications 128 to logically obtain, process, and present the relatively amorphous data 132 from the database 130.

The server 102 is implemented using one or more actual and/or virtual computing systems that collectively provide the dynamic application platform 110 for generating the virtual applications 128. For example, the server 102 may be implemented using a cluster of actual and/or virtual servers operating in conjunction with each other, typically in association with conventional network communications, cluster management, load balancing and other features as appropriate. The server 102 operates with any sort of conventional processing hardware 104, such as a processor 105, memory 106, input/output features 107 and the like. The input/output features 107 generally represent the interface(s) to networks (e.g., to the network 145, or any other local area, wide area or other network), mass storage, display devices, data entry devices and/or the like. The processor 105 may be implemented using any suitable processing system, such as one or more processors, controllers, microprocessors, microcontrollers, processing cores and/or other computing resources spread across any number of distributed or integrated systems, including any number of “cloud-based” or other virtual systems. The memory 106 represents any non-transitory short or long term storage or other computer-readable media capable of storing programming instructions for execution on the processor 105, including any sort of random access memory (RAM), read only memory (ROM), flash memory, magnetic or optical mass storage, and/or the like. The computer-executable programming instructions, when read and executed by the server 102 and/or processor 105, cause the server 102 and/or processor 105 to create, generate, or otherwise facilitate the application platform 110 and/or virtual applications 128 and perform one or more additional tasks, operations, functions, and/or processes described herein. It should be noted that the memory 106 represents one suitable implementation of such computer-readable media, and alternatively or additionally, the server 102 could receive and cooperate with external computer-readable media that is realized as a portable or mobile component or application platform, e.g., a portable hard drive, a USB flash drive, an optical disc, or the like.

The application platform 110 is any sort of software application or other data processing engine that generates the virtual applications 128 that provide data and/or services to the client devices 140. In a typical embodiment, the application platform 110 gains access to processing resources, communications interfaces and other features of the processing hardware 104 using any sort of conventional or proprietary operating system 108. The virtual applications 128 are typically generated at run-time in response to input received from the client devices 140. For the illustrated embodiment, the application platform 110 includes a bulk data processing engine 112, a query generator 114, a search engine 116 that provides text indexing and other search functionality, and a runtime application generator 120. Each of these features may be implemented as a separate process or other module, and many equivalent embodiments could include different and/or additional features, components or other modules as desired.

The runtime application generator 120 dynamically builds and executes the virtual applications 128 in response to specific requests received from the client devices 140. The virtual applications 128 are typically constructed in accordance with the tenant-specific metadata 138, which describes the particular tables, reports, interfaces and/or other features of the particular application 128. In various embodiments, each virtual application 128 generates dynamic web content that can be served to a browser or other client program 142 associated with its client device 140, as appropriate.

The runtime application generator 120 suitably interacts with the query generator 114 to efficiently obtain multi-tenant data 132 from the database 130 as needed in response to input queries initiated or otherwise provided by users of the client devices 140. In a typical embodiment, the query generator 114 considers the identity of the user requesting a particular function (along with the user's associated tenant), and then builds and executes queries to the database 130 using system-wide metadata 136, tenant specific metadata 138, pivot tables 134, and/or any other available resources. The query generator 114 in this example therefore maintains security of the common database 130 by ensuring that queries are consistent with access privileges granted to the user and/or tenant that initiated the request. In this manner, the query generator 114 suitably obtains requested subsets of data 132 accessible to a user and/or tenant from the database 130 as needed to populate the tables, reports or other features of the particular virtual application 128 for that user and/or tenant.

Still referring to FIG. 1, the data processing engine 112 performs bulk processing operations on the data 132 such as uploads or downloads, updates, online transaction processing, and/or the like. In many embodiments, less urgent bulk processing of the data 132 can be scheduled to occur as processing resources become available, thereby giving priority to more urgent data processing by the query generator 114, the search engine 116, the virtual applications 128, etc.

In exemplary embodiments, the application platform 110 is utilized to create and/or generate data-driven virtual applications 128 for the tenants that they support. Such virtual applications 128 may make use of interface features such as custom (or tenant-specific) screens 124, standard (or universal) screens 122 or the like. Any number of custom and/or standard objects 126 may also be available for integration into tenant-developed virtual applications 128. As used herein, “custom” should be understood as meaning that a respective object or application is tenant-specific (e.g., only available to users associated with a particular tenant in the multi-tenant system) or user-specific (e.g., only available to a particular subset of users within the multi-tenant system), whereas “standard” or “universal” applications or objects are available across multiple tenants in the multi-tenant system. For example, a virtual CRM application may utilize standard objects 126 such as “account” objects, “opportunity” objects, “contact” objects, or the like. The data 132 associated with each virtual application 128 is provided to the database 130, as appropriate, and stored until it is requested or is otherwise needed, along with the metadata 138 that describes the particular features (e.g., reports, tables, functions, objects, fields, formulas, code, etc.) of that particular virtual application 128. For example, a virtual application 128 may include a number of objects 126 accessible to a tenant, wherein for each object 126 accessible to the tenant, information pertaining to its object type along with values for various fields associated with that respective object type are maintained as metadata 138 in the database 130. In this regard, the object type defines the structure (e.g., the formatting, functions and other constructs) of each respective object 126 and the various fields associated therewith.

Still referring to FIG. 1, the data and services provided by the server 102 can be retrieved using any sort of personal computer, mobile telephone, tablet or other network-enabled client device 140 on the network 145. In an exemplary embodiment, the client device 140 includes a display device, such as a monitor, screen, or another conventional electronic display capable of graphically presenting data and/or information retrieved from the multi-tenant database 130. Typically, the user operates a conventional browser application or other client program 142 executed by the client device 140 to contact the server 102 via the network 145 using a networking protocol, such as the hypertext transport protocol (HTTP) or the like. The user typically authenticates his or her identity to the server 102 to obtain a session identifier (“SessionID”) that identifies the user in subsequent communications with the server 102. When the identified user requests access to a virtual application 128, the runtime application generator 120 suitably creates the application at run time based upon the metadata 138, as appropriate. As noted above, the virtual application 128 may contain Java, ActiveX, or other content that can be presented using conventional client software running on the client device 140; other embodiments may simply provide dynamic web or other content that can be presented and viewed by the user, as desired. In various embodiments, application 128 embodies the functionality of a collaboration solution such as a social networking system.

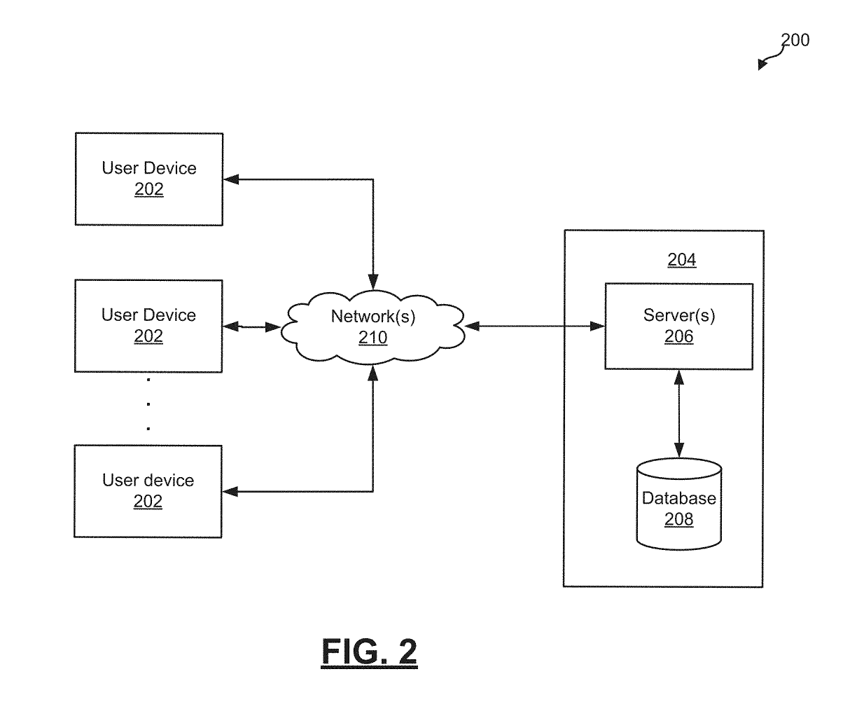

FIG. 2 is a block diagram of an example computing environment 200 that can be used to implement aspects of the current subject matter. The example system includes one or more clients, e.g., user devices 202, that access information feeds from a software as a service (SAAS) system 204. The SAAS system 204 includes a server 206 that can access an enterprise network database 208 and provide information feeds to a user device 202 via a network 210. An example SAAS system includes the Salesforce Chatter system.

The example SAAS system 204 provides a web interface for use by the user devices. The user devices may operate a web browser to access information feeds from the enterprise network database 208 via the web interface and the server 206.

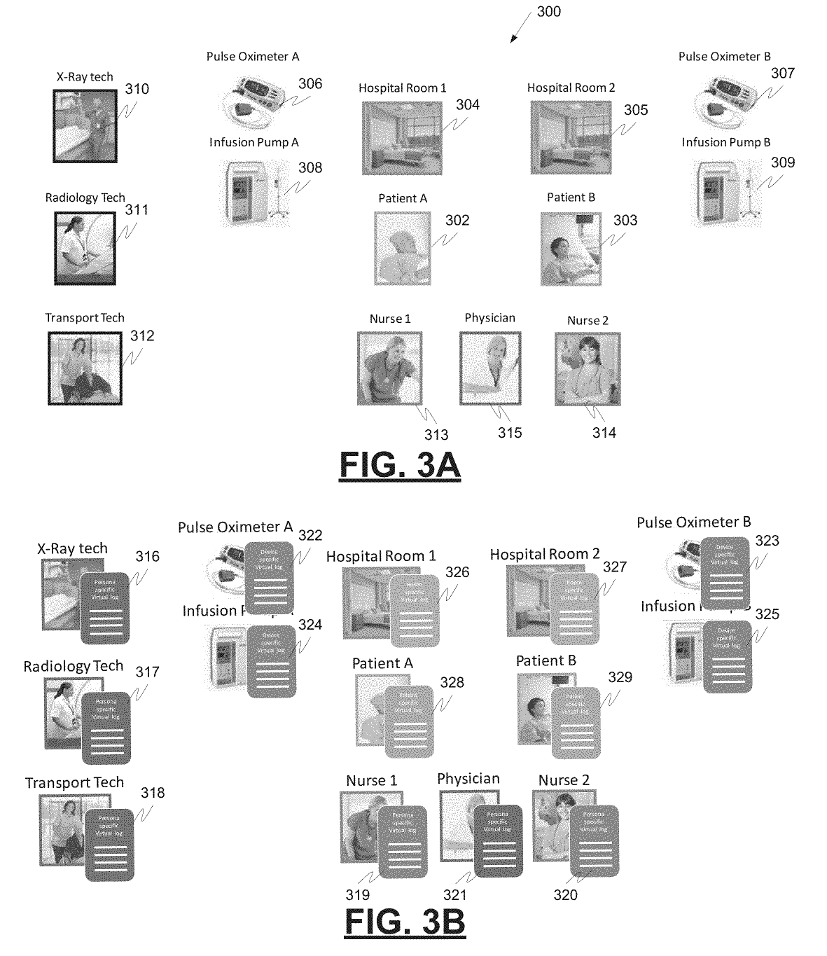

FIG. 3A is a diagram depicting example personas, devices, and notification targets in an example patient care enterprise environment in which an example enterprise social networking system may be used. The example patient care enterprise environment 300 is a patient care setting such as a hospital environment. The example hospital environment 300 includes patients 302, 303 (e.g., Patient A and Patient B) for whom care is provided, hospital rooms 304, 305 (e.g., Hospital Room 1 and Hospital Room 2) that may be assigned to patients when care is rendered, and medical devices 306, 307, 308, 309 (e.g., Pulse Oximeter A, Pulse Oximeter B, Infusion Pump A, and Infusion Pump B) that may be used in hospital rooms for patient care. The example hospital environment 300 also includes personas, such as an X-Ray technician 310, a Radiology Technician 311, a Transport Technician 312, Nurses 313, 314 (e.g., Nurse 1 and Nurse 2), and a Physician 315, who deliver patient care to the patients.

When there is an event associated with a patient, an instrumentality for rendering patient care (e.g., hospital room or medical device), or a persona for rendering patient care (e.g., a technician, nurse, or physician), the event can be recorded in an event log for the enterprise in an enterprise network database. User groups can be created that allow subscribers to the user group to view events related to the user group.

As an example, a Patient A user group can be created and a virtual log associated with the Patient A user group can be generated from the enterprise event log that only provides a view of events that are associated with Patient A. Subscribers to the Patient A user group can receive an information feed that provides a view of events associated with Patient A. Similarly, user groups can be created and associated virtual logs generated for the other patients, instrumentalities used for rendering patient care, and personas that render patient care.

FIG. 3B is a diagram depicting example personas, instrumentalities, and patients in an example patient care enterprise environment and user group specific virtual logs that can be generated to track events associated with the personas, instrumentalities, and patients. Each of the personas has an associated persona specific virtual log that provides a view of events that are associated with the specific persona. For example, the X-Ray technician persona has a persona specific virtual log 316, the Radiology Technician persona has a persona specific virtual log 317, the Transport Technician persona has a persona specific virtual log 318, the Nurse 1 persona has a persona specific virtual log 319, the Nurse 2 persona has a persona specific virtual log 320, and the Physician persona has a persona specific virtual log 321.

Similarly, each of the instrumentalities has an instrumentality specific virtual log. For example, the Pulse Oximeter A device has a device specific virtual log 322, the Pulse Oximeter B device has a device specific virtual log 323, the Infusion Pump A has a device specific virtual log 324, the Infusion Pump B has a device specific virtual log 325, the Hospital Room 1 has a room specific virtual log 326, and the Hospital Room 2 has a room specific virtual log 327.

Likewise, each of the patients has a patient specific virtual log. For example, Patient A has a patient specific virtual log 328and Patient B device has a patient specific virtual log 329.

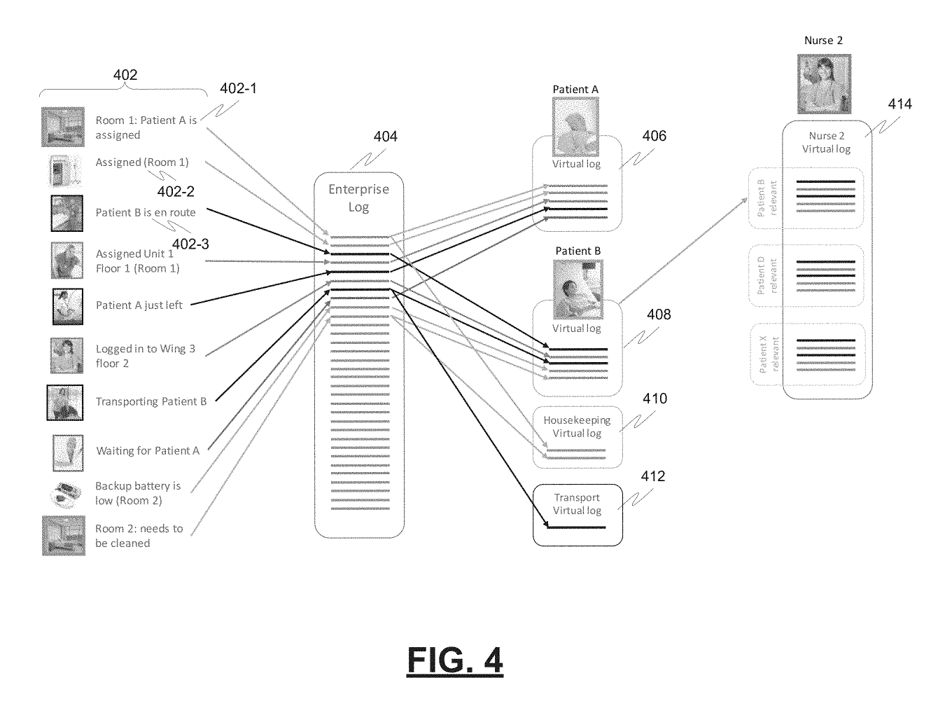

FIG. 4 is a relationship diagram illustrating an example relationship between events 402, an enterprise log 404, and user group specific virtual logs 406, 408, 410, 412, 414. In the example relationship diagram, events 402 are entered in the enterprise log 404 as they occur. Events entered in the enterprise log can subsequently be included in one or more virtual logs depending on the persona to which the event relates.

As an example, a first event 402-1, Patient A is assigned to Room 1, is entered in the first line of the enterprise log 404. That first entry in the enterprise log 404 may then be included in a Patient A virtual log 406 and a Housekeeping virtual log 410 because it pertains to both the Patient A persona and the Housekeeping persona. A second event 402-2, an infusion pump being assigned to Room 1, is entered in the next line of the enterprise log 404. That second entry in the enterprise log 404 may then be included in a Patient A virtual log 406 because it pertains to the Patient A persona. A third event 402-3, Patient B is en route, is entered in the next line of the enterprise log 404. That third entry in the enterprise log 404 may then be included in a Patient B virtual log 408 and a Transport virtual log 412 because it pertains to both the Patient B persona and the Transport persona. Additional events 402 may be entered in the enterprise log 404. Those additional entries in the enterprise log 404 may then be included in one or more virtual logs 406, 408, 410, 412, 414 based on the persona(s) to which the events pertain.

FIG. 4 also illustrates an example virtual log that may be derived indirectly from the enterprise log 404. In this example, a Nurse 2 virtual log 414 is derived from one or more other virtual logs and may be derived directly or indirectly from the enterprise log 404. The Nurse 2 persona may be assigned to a patient care team for multiple patients, (e.g., patient B, patient D, and patient X). Thus, the Nurse 2 virtual log 414 may include events from the enterprise log 404 relating to each of patient B, patient D, and patient X. The virtual log may be formed by excluding from view events from the enterprise log that do not pertain to the associated group.

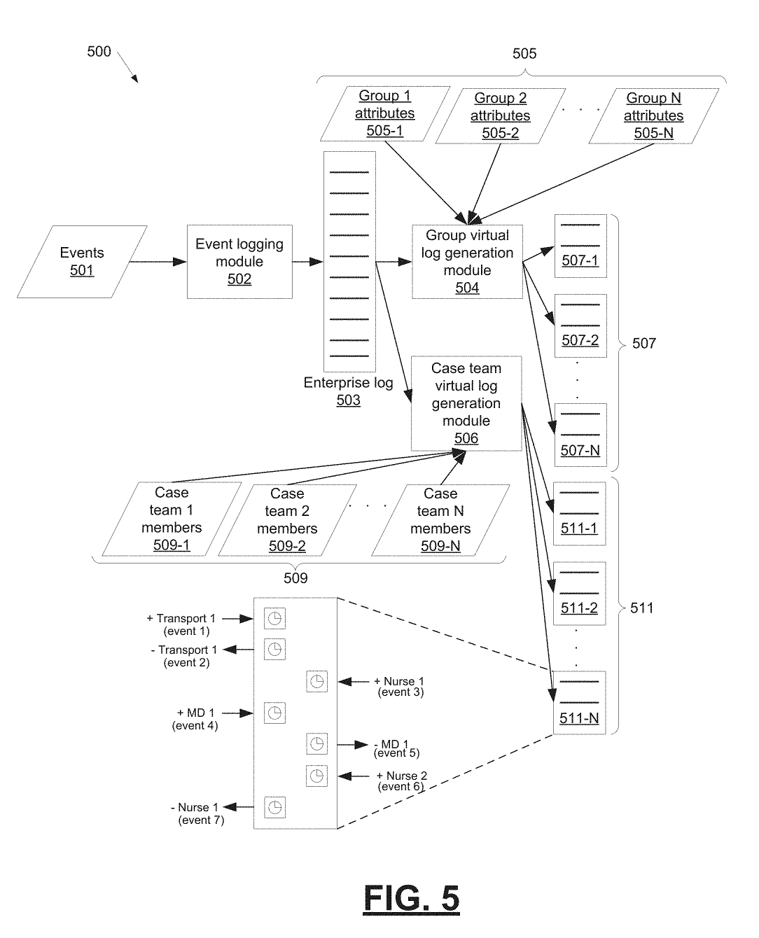

FIG. 5 is a block diagram depicting an example enterprise networking system 500 for generating group virtual logs and case team virtual logs from which a notification feed can be provided. The example system 500 includes an event logging module 502, a group virtual log generation module 504, and a case team virtual log generation module 506.

The example event logging module 502 is configured to receive enterprise event information 501 and to log the event information 501 in an enterprise log 503 as the event information is received. The example enterprise log 503 contains a listing of all enterprise events.

The example group virtual log generation module 504 is configured to identify entries from the enterprise log 503 to include in a group virtual log 507. The example group virtual log generation module 504 is configured to generate a virtual log for each of a plurality of defined groups from the enterprise log 503.

As an example, a virtual log 507-1 can be generated for an example group with Group 1 attributes 505-1. If the example Group 1 attributes 505-1 identified Group 1 as relating to events involving patient A, then all events from the enterprise log 503 relating to the patient A would be included in the example virtual log 507-1. A subscriber to the example Group 1 for patient A could receive a feed that contains a listing of all events relating to patient A.

As another example, a virtual log 507-2 can be generated for an example group with Group 2 attributes 505-2. If the example Group 2 attributes 505-2 identified Group 2 as relating to events involving Housekeeping, then all events from the enterprise log 503 relating to Housekeeping would be included in the example virtual log 507-2. A subscriber to the example Group 2 for Housekeeping could receive a feed that contains a listing of all events relating to Housekeeping.

A subscriber to one of the example groups however may receive too much information in its feed if it receives a feed that contains a listing of all events relating to a group to which it is subscribed. The example case team virtual log generation module 506 is configured to generate a case team virtual log 511 from which a more targeted feed may be generated for a subscriber to the case team. The example case team virtual log generation module 506 is configured to generate a virtual log for each of a plurality of defined case teams from the enterprise log 503.

As an example, a case team virtual log 511-1 can be generated for example members of a first case team 509-1. If the example first case team is related to events involving a patient B, then all events from the enterprise log 503 relating to the patient B and involving case team members would be included in the example virtual log 511-1 including the time when a case team member joins or leaves the case team. This may allow a case team member for the patient B case team to receive a more targeted feed that contains a listing of all events relating to patient B that occurred while the case team member was a member of the case team.

As an example, a first transport technician may receive a feed item when it is assigned to the patient B case team (event 1) and when it completes its transport task and is removed from the patient B case team (event 2). If any events involving patient B were to occur between when the first transport technician joined the case team and left the case team, then the first transport technician could receive a feed item regarding that event. The transport technician would not receive notifications regarding events occurring before or after it was a member of the case team.

In another example, a first nurse may receive a feed item when it is assigned to the patient B case team (event 3) and when it completes its shift and is removed from the patient B case team (event 7). The first nurse may receive a feed item notifying it of events involving patient B occurring between when the first nurse joined the case team and left the case team. In this example, the first nurse may receive a feed item when a doctor arrives to perform a procedure (event 4), when the doctor finishes the procedure (event 5), and when a second nurse joins the case team (event 6). The first nurse would not receive notifications regarding events occurring before or after it was a member of the case team.

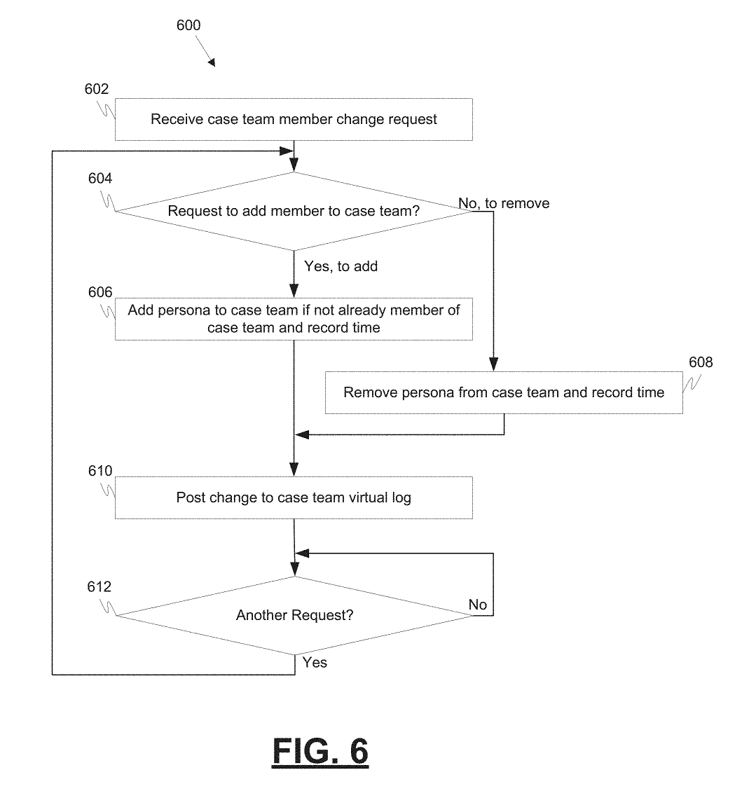

FIG. 6 is a process flow chart depicting an example process 600 for adding or removing members from a case team in an enterprise networking system. This example process 600 (and other processes described herein) may be implemented at least partially with a multi-tenant database system, e.g., by one or more processors configured to receive or retrieve information, process the information, store results, and transmit the results. This example process 600 may also be implemented at least partially with a single tenant database system.

At operation 602, the enterprise networking system receives a request to change the membership of a case team relating to a case. The case may be related to the treatment of a patient, for example. The request may be to add a member to a team, for example, as the result of a persona beginning a work shift or a persona being assigned a task to perform relating to the case. The request may also be to remove a member from the team, for example, as the result of a persona completing a work shift or a persona having completed a task relating to the case.

A determination is made regarding whether the request is to add a member to the case team or to remove a member from the case team (decision 604). If the request is to add a member to the case team (yes at decision 604), then a member is added to the case team if not already a member of the case team (operation 606) and the change is added to the case team virtual log (operation 610). If the request is to remove a member from the case team (no at decision 604), then the member is removed from the case team (operation 608) and the change is added to the case team virtual log (operation 610).

After the change is posted to the case team virtual log, the system waits for additional change requests (decision 612). When an additional change request is sensed (yes at decision 612), a determination is made regarding whether the request is to add a member to the case team or to remove a member from the case team (decision 604).

Members of a case team may include devices, rooms, or other instrumentalities in addition to personas. The process for adding members other than personas may be the same as or similar to the example process 600.

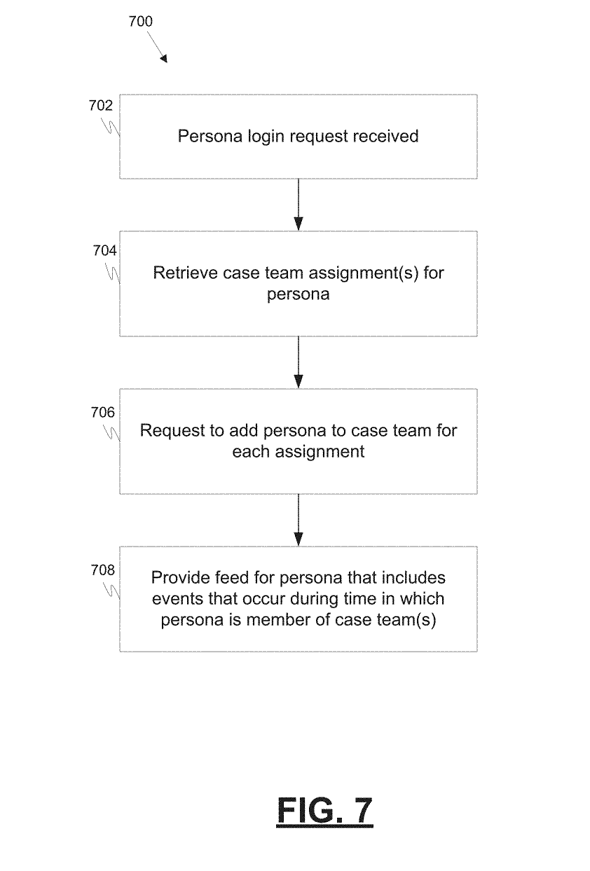

FIG. 7 is a process flow chart depicting an example process in an enterprise networking system for providing a targeted feed for a persona. At operation 702, a persona login request is received. This may occur, for example, when a nurse logs in for duty. The enterprise networking system may retrieve the case team assignments for the persona (operation 704). As an example, a nurse may be assigned to three patients. The enterprise networking system in this example would retrieve the nurse's assignment to the case team for these three patients. The enterprise networking system may then request to add the persona (e.g., the nurse) to the case team for each assignment (e.g., each patient's case team) (operation 706).

The enterprise networking system may provide feed items for the persona that includes events that occur during the time in which the persona is a member of the case team(s) (operation 708). For example, if the persona (e.g., a nurse) is a member of a case team for three patients (e.g., patient B, patient D, and patient X), then the persona can be provided a feed (e.g., a virtual log) that includes events occurring with patient B while the nurse is a member of patient B's case team, events occurring with patient D while the nurse is a member of patient D's case team, and events occurring with patient X while the nurse is a member of patient X's case team. The feed may be organized by case, chronologically, or by some other methodology. Thus, the nurse, in this example, is provided a notification view that includes a role-specific view for each case team on which the nurse is a member.

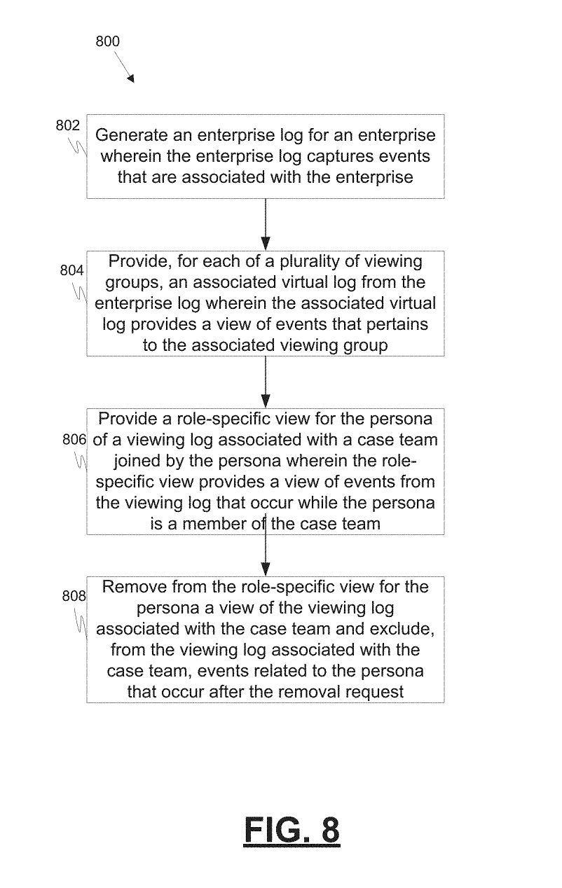

FIG. 8 is a process flow chart depicting another example process in an enterprise networking system for providing a targeted feed for a persona. At operation 802, a database system in an enterprise networking system generates an enterprise log for an enterprise wherein the enterprise log captures events that are associated with the enterprise.

At operation 804, the database system provides, for each of a plurality of persona-defined viewing groups, an associated virtual log from the enterprise log wherein the associated virtual log provides a view of events from the enterprise log that pertains to subscribers to the persona-defined viewing group with which the associated virtual log is associated. The associated virtual log may be generated by evaluating each event in the enterprise log to determine if it is relevant to a persona-defined viewing group and removing events that are not relevant to the persona-defined viewing group.

At operation 806, the database system provides, responsive to a joining request for a persona to become a member of a case team, a role-specific view for the persona of a viewing log associated with the case team wherein the viewing log provides a view of events from the enterprise log that pertains to members of the case team and that occur while the member to which the event pertains is a member of the case team. The role-specific view for the persona provides a view of events from the viewing log that occur while the persona is a member of the case team.

At operation 808, the database system, responsive to a removal request for the persona to be removed as a member of the case team, removes from the role-specific view for the persona a view of the viewing log associated with the case team and excludes, from the viewing log associated with the case team, events related to the persona that occur after the removal request (operation 808).

Disclosed herein are systems and methods for customizing a user's information feed to focus the information feed to feed items that may be more relevant to the user. The apparatus, systems, techniques and articles described can provide a virtual log for a persona on a case team that only includes feed items that occur while the persona is a member of the case team.

In one embodiment, a method of providing role specific notification views in a database system associated with an enterprise networking system is provided. The method comprises generating, using the database system, an enterprise log for an enterprise wherein the enterprise log captures events that are associated with the enterprise; responsive to a joining request for a persona to become a member of a case team, providing, by the database system, a role-specific view for the persona of a viewing log associated with the case team wherein the viewing log provides a view of events from the enterprise log that pertains to members of the case team and that occur while the member to which the event pertains is a member of the case team and wherein the role-specific view for the persona provides a view of events from the viewing log that occur while the persona is a member of the case team; and sending the role-specific view for the persona to a web browser.

These aspects and other embodiments may include one or more of the following features. The enterprise may comprise a patient care enterprise and the case team may comprise a patient care team. Events from the enterprise log that pertains to members of the case team may comprise events from the enterprise log that pertains to a patient or one or more members of the patient care team for the patient. The patient care team members may comprise one or more of a room, a medical device, a nurse, a physician, a medical technician, and a non-medically trained employee of the patient care enterprise. The method may further comprise responsive to a removal request for the persona to be removed as a member of the case team, removing, by the database system, from the role-specific view for the persona, a view of the viewing log associated with the case team and excluding events related to the persona that occur after the removal request from the viewing log associated with the case team. The method may further comprise for each of a plurality of persona-defined viewing groups, providing, by the database system, an associated virtual log from the enterprise log wherein the associated virtual log provides a view of events from the enterprise log that pertains to subscribers to the persona-defined viewing group with which the associated virtual log is associated. Providing an associated virtual log may comprise evaluating each event in the enterprise log to determine if it is relevant to a persona-defined viewing group and removing events that are not relevant to the persona-defined viewing group.

In another embodiment, a computer-implemented method of providing role specific notification views in an enterprise social networking system is provided. The method comprises receiving, at a server in the enterprise social networking system, a connection request for a persona; retrieving, using the server, one or more case team assignments for the persona; for each case team assignment for the persona, adding, by the server, the persona to the case team for the assignment; for each case team assignment for the persona, retrieving, by the server, a role-specific view for the persona of a viewing log associated with the assigned case team wherein the viewing log provides a view of events that pertains to members of the case team and that occur while the members are members of the case team and wherein the role-specific view for the persona provides a view of events from the viewing log that occur while the persona is a member of the case team; and providing a notification view, by the server, that includes the role-specific view for each case team assignment for the persona.

These aspects and other embodiments may include one or more of the following features. The enterprise social networking system may comprise an enterprise social networking system for a patient care enterprise and the case team assignment may comprise an assignment on a patient care team. The events that pertains to members of the case team may comprise events that pertains to a patient and one or more members of the patient care team for the patient. The patient case team members may comprise one or more of a room, a medical device, a nurse, a physician, a medical technician, and a non-medically trained employee of the patient care enterprise. The method may further comprise receiving a removal request to remove the persona from a case team; removing the persona from the case team; and not allowing a role-specific view for the persona of the viewing log associated with the unassigned case team. The method may further comprise excluding, from the viewing log associated with the unassigned case team, events related to the persona that occur after the removal request. Receiving a connection request may comprise receiving a connection request via a web based interface and providing a notification view may comprise providing a notification view via the web based interface. The web based interface may comprise a web browser.

In another embodiment, an enterprise social networking system is provided. The enterprise social networking system comprises one or more processors and non-transient computer readable media coupled to the one or more processors wherein the non-transient computer readable media embodies programming instructions configurable to perform a method. The method comprises receiving, at a server in the enterprise social networking system, a connection request for a persona; retrieving, using the server, one or more case team assignments for the persona; for each case team assignment for the persona, adding, by the server, the persona to the case team for the assignment; for each case team assignment for the persona, retrieving, by the server, a role-specific view for the persona of a viewing log associated with the assigned case team wherein the viewing log provides a view of events that pertains to members of the case team and that occur while the members are members of the case team and wherein the role-specific view for the persona provides a view of events from the viewing log that occur while the persona is a member of the case team; and providing a notification view, by the server, that includes the role-specific view for each case team assignment for the persona.

These aspects and other embodiments may include one or more of the following features. The enterprise social networking system may comprise an enterprise social networking system for a patient care enterprise and the case team assignment may comprise an assignment on a patient care team. The events that pertains to members of the case team may comprise events that pertains to a patient and one or more members of the patient care team for the patient. The patient care team members may comprise one or more of a room, a medical device, a nurse, a physician, a medical technician, and a non-medically trained employee of the patient care enterprise. The method may further comprise receiving a removal request to remove the persona from a case team, removing the persona from the case team, not allowing a role-specific view for the persona of the viewing log associated with the unassigned case team, and excluding, from the viewing log associated with the unassigned case team, events related to the persona that occur after the removal request. Receiving a connection request may comprise receiving a connection request via a web based interface, providing a notification view may comprise providing a notification view via the web based interface, and the web based interface may comprise a web browser.

The foregoing description is merely illustrative in nature and is not intended to limit the embodiments of the subject matter or the application and uses of such embodiments. Furthermore, there is no intention to be bound by any expressed or implied theory presented in the technical field, background, or the detailed description. As used herein, the word “exemplary” means “serving as an example, instance, or illustration.” Any implementation described herein as exemplary is not necessarily to be construed as preferred or advantageous over other implementations, and the exemplary embodiments described herein are not intended to limit the scope or applicability of the subject matter in any way.

For the sake of brevity, conventional techniques related to object models, web pages, multi-tenancy, cloud computing, on-demand applications, and other functional aspects of the systems (and the individual operating components of the systems) may not be described in detail herein. In addition, those skilled in the art will appreciate that embodiments may be practiced in conjunction with any number of system and/or network architectures, data transmission protocols, and device configurations, and that the system described herein is merely one suitable example. Furthermore, certain terminology may be used herein for the purpose of reference only, and thus is not intended to be limiting. For example, the terms “first,” “second” and other such numerical terms do not imply a sequence or order unless clearly indicated by the context.

Embodiments of the subject matter may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. In practice, one or more processing systems or devices can carry out the described operations, tasks, and functions by manipulating electrical signals representing data bits at accessible memory locations, as well as other processing of signals. The memory locations where data bits are maintained are physical locations that have particular electrical, magnetic, optical, or organic properties corresponding to the data bits. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices. When implemented in software or firmware, various elements of the systems described herein are essentially the code segments or instructions that perform the various tasks. The program or code segments can be stored in a processor-readable medium or transmitted by a computer data signal embodied in a carrier wave over a transmission medium or communication path. The “processor-readable medium” or “machine-readable medium” may include any non-transitory medium that can store or transfer information. Examples of the processor-readable medium include an electronic circuit, a semiconductor memory device, a ROM, a flash memory, an erasable ROM (EROM), a floppy diskette, a CD-ROM, an optical disk, a hard disk, a fiber optic medium, a radio frequency (RF) link, or the like. The computer data signal may include any signal that can propagate over a transmission medium such as electronic network channels, optical fibers, air, electromagnetic paths, or RF links. The code segments may be downloaded via computer networks such as the Internet, an intranet, a LAN, or the like. In this regard, the subject matter described herein can be implemented in the context of any computer-implemented system and/or in connection with two or more separate and distinct computer-implemented systems that cooperate and communicate with one another. In one or more exemplary embodiments, the subject matter described herein is implemented in conjunction with a virtual customer relationship management (CRM) application in a multi-tenant environment.

While at least one exemplary embodiment has been presented, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application. Accordingly, details of the exemplary embodiments or other limitations described above should not be read into the claims absent a clear intention to the contrary.

What is claimed is:

1. A method of providing role specific notification views in a database system associated with an enterprise networking system, the method comprising:
generating, using the database system, an enterprise log for an enterprise, the enterprise log capturing events that are associated with the enterprise;
responsive to a joining request for a persona to become a member of a case team, providing, by the database system, a role-specific view for the persona of a viewing log associated with the case team wherein the viewing log provides a view of events from the enterprise log that pertains to members of the case team and that occur while the member to which the event pertains is a member of the case team, the role-specific view for the persona providing a view of events from the viewing log that occur while the persona is a member of the case team; and
sending the role-specific view for the persona to a web browser.

2. The method of claim 1, wherein the enterprise comprises a patient care enterprise and wherein the case team comprises a patient care team.

3. The method of claim 2, wherein events from the enterprise log that pertains to members of the case team comprise events from the enterprise log that pertains to a patient or one or more members of the patient care team for the patient.

4. The method of claim 3, wherein the patient care team members comprise one or more of a room, a medical device, a nurse, a physician, a medical technician, and a non-medically trained employee of the patient care enterprise.

5. The method of claim 1, further comprising:
responsive to a removal request for the persona to be removed as a member of the case team, removing, by the database system, from the role-specific view for the persona, a view of the viewing log associated with the case team and excluding events related to the persona that occur after the removal request from the viewing log associated with the case team.

6. The method of claim 1, further comprising:
for each of a plurality of persona-defined viewing groups, providing, by the database system, an associated virtual log from the enterprise log wherein the associated virtual log provides a view of events from the enterprise log that pertains to subscribers to the persona-defined viewing group with which the associated virtual log is associated.

7. The method of claim 6, wherein providing an associated virtual log comprises:
evaluating each event in the enterprise log to determine if it is relevant to a persona-defined viewing group; and
removing events that are not relevant to the persona-defined viewing group.

8. A computer-implemented method of providing role specific notification views in an enterprise social networking system, the method comprising:
receiving, at a server in the enterprise social networking system, a connection request for a persona;
retrieving, using the server, one or more case team assignments for the persona;
for each case team assignment for the persona, adding, by the server, the persona to the case team for the assignment;
for each case team assignment for the persona, retrieving, by the server, a role-specific view for the persona of a viewing log associated with the assigned case team wherein the viewing log provides a view of events that pertains to members of the case team and that occur while the members are members of the case team, the role-specific view for the persona providing a view of events from the viewing log that occur while the persona is a member of the case team; and
providing a notification view, by the server, that includes the role-specific view for each case team assignment for the persona.

9. The method of claim 8, wherein the enterprise social networking system comprises an enterprise social networking system for a patient care enterprise and wherein the case team assignment comprises an assignment on a patient care team.

10. The method of claim 9, wherein the events that pertains to members of the case team comprise events that pertains to a patient and one or more members of the patient care team for the patient.

11. The method of claim 10, wherein the patient case team members comprise one or more of a room, a medical device, a nurse, a physician, a medical technician, and a non-medically trained employee of the patient care enterprise.

12. The method of claim 8, further comprising:
receiving a removal request to remove the persona from a case team;
removing the persona from the case team; and
preventing a role-specific view for the persona of the viewing log associated with the unassigned case team.

13. The method of claim 12, further comprising:
excluding, from the viewing log associated with the unassigned case team, events related to the persona that occur after the removal request.

14. The method of claim 8, wherein receiving a connection request comprises receiving a connection request via a web based interface and wherein providing a notification view comprises providing a notification view via the web based interface.

15. An enterprise social networking system comprising one or more processors and non-transient computer readable media coupled to the one or more processors, the non-transient computer readable media embodying programming instructions configurable to perform a method, the method comprising:
receiving, at a server in the enterprise social networking system, a connection request for a persona;
retrieving, using the server, one or more case team assignments for the persona;
for each case team assignment for the persona, adding, by the server, the persona to the case team for the assignment;
for each case team assignment for the persona, retrieving, by the server, a role-specific view for the persona of a viewing log associated with the assigned case team wherein the viewing log provides a view of events that pertains to members of the case team and that occur while the members are members of the case team, the role-specific view for the persona providing a view of events from the viewing log that occur while the persona is a member of the case team; and
providing a notification view, by the server, that includes the role-specific view for each case team assignment for the persona.

16. The enterprise social networking system of claim 15, wherein the enterprise social networking system comprises an enterprise social networking system for a patient care enterprise and wherein the case team assignment comprises an assignment on a patient care team.

17. The enterprise social networking system of claim 16, wherein the events that pertains to members of the case team comprise events that pertains to a patient and one or more members of the patient care team for the patient.

18. The enterprise social networking system of claim 17, wherein the patient care team members comprise one or more of a room, a medical device, a nurse, a physician, a medical technician, and a non-medically trained employee of the patient care enterprise.

19. The enterprise social networking system of claim 15, wherein the method further comprises:
receiving a removal request to remove the persona from a case team;
removing the persona from the case team;
preventing a role-specific view for the persona of the viewing log associated with the unassigned case team; and
excluding, from the viewing log associated with the unassigned case team, events related to the persona that occur after the removal request.

20. The enterprise social networking system of claim 15, wherein:
receiving a connection request comprises receiving a connection request via a web based interface;
providing a notification view comprises providing a notification view via the web based interface; and
the web based interface comprises a web browser.
